SEQUENCE LISTING (6 sequences)

SEQ ID NO: 1 - DNA, 411 bp, Klebsiella pneumoniae
  CDS (1)..(411)
gtggctaccaagcacttcaccctgaagtctgacgttctgttcaacttcaacaaagctaccctgaaaccggaaggtcagcaggctctggatcagctgtacactcagctgagcaacatggatccgaaagacggttccgctgttgttctgggctacaccgaccgcatcggttccgaagcttacaaccagcagctgtctgagaaacgtgctcagtccgtcgttgactacctggttgctaaaggcatcccggctggcaaaatctccgctcgcggcatgggtgaatccaacccggttactggcaacacctgtgacaacgtgaaagctcgcgctgccctgatcgattgcctggctccggatcgtcgtgtagagatcgaagttaaaggctacaaagaagttgtaactcagcctcaggct

SEQ ID NO: 2 - protein, 137 aa, Klebsiella pneumoniae
VATKHFTLKSDVLFNFNKATLKPEGQQALDQLYTQLSNMDPKDGSAVVLGYTDRIGSEAYNQQLSEKRAQSVVDYLVAKGIPAGKISARGMGESNPVTGNTCDNVKARAALIDCLAPDRRVEIEVKGYKEVVTQPQA

SEQ ID NO: 3 - DNA, 29 bp, Artificial sequence
  Description of artificial sequence primer sequence derived from OmpA P40 protein from Klebsiella pneumoniae (sense)
gcgaattcgtggctaccaagcacttcacc

SEQ ID NO: 4 - DNA, 44 bp, Artificial sequence
  Description of artificial sequence primer sequence derived from OmpA P40 protein from  Klebsiella pneumoniae (antisense)
cgaagcttagtggtggtggtggtggtgttccggagccggagccg

SEQ ID NO: 5 - protein, 9 aa, Artificial sequence
  Description of artificial sequence 9 amino acid sequence upstream from leader peptide of tryptophane operon.
MKAIFVLNA

SEQ ID NO: 6 - protein, 16 aa, Syncytial respiratory virus (SRV)
  Description of the sequence G5 peptide corresponding to aa144-159 fragment of human type A SRV G protein
SKPTTKQRQNKPPNKP

(19) United States
(12) Patent Application Publication
(10) Pub. No.: US 2004/0014661 A1
(43) Pub. Date: Jan. 22, 2004

(54) Periplasmic domain of an enterobacterium omp protein and its use as carrier or adjuvant

(76) Inventors: Liliane Goetsch, Ayze (FR); Jean-Francois Haeuw, Saint Julien en Genevois (FR); Alain Robert, Annemasse (FR)

Correspondence Address: G. Patrik Sage, The Firm of Hueschen and Sage, 715 The H Building, 310 East Michigan Avenue, Kalamazoo, MI, 49007, US

(21) Appl. No.: 10/432,056

(22) PCT Filed: Nov. 16, 2001
(86) PCT No.: PCT/FR01/03596

(30) Foreign Application Priority Data
Nov. 17, 2000 (FR) 00/14909

(51) Int. Cl. A61K 48/00; A61K 39/02

(52) U.S. Cl. 514/12; 514/44; 424/190.1

(57) ABSTRACT

The invention concerns pharmaceutical composition comprising in a pharmaceutically acceptable medium at least a peptide derived from a periplasmic domain of an enterobacterium Omp protein or a nucleic construct coding for said peptide, as well as the use of such a peptide or such a nucleic construct for the preparation of a vaccine for prophylactic or therapeutic treatment of viral, bacterial, parasitic or fungal infections, or for preparing a vaccine designed for the prophylactic or therapeutic treatment of cancers.

The invention relates to a pharmaceutical composition comprising, in a pharmaceutically acceptable medium, at least one peptide obtained from the periplasmic domain of an enterobacterium Omp protein or a nucleic construct encoding said peptide, and the use of such a peptide or such a nucleic construct for the preparation of a vaccine intended for the prophylactic or therapeutic treatment of viral, bacterial, parasitic or fungal infections, or for the preparation of a vaccine intended for the prophylactic or therapeutic treatment of cancers.

Vaccination is an effective means of preventing or reducing viral or bacterial infections. The success of vaccination campaigns in these fields have made it possible to extend the vaccine concept which was thus far used in the field of infectiology to the fields of cancer and autoimmune diseases.

The development of perfectly defined vaccines free of marked side effects requires the use of low molecular mass vaccine antigens such as peptides or oligosaccharides. These antigens of low mass, but also some antigens of higher molecular mass such as bacterial wall polysaccharides, cannot alone induce a lasting and intense immune response. It is essential to attach these antigens, by chemical means or by genetic engineering, to carrier proteins.

The carrier proteins currently used are of two types:

tetanus and diphtheria toxoids: the too frequent use of these carrier proteins risks running counter to an intense response against the hapten and risks posing problems of immunotoxicology,

extracts of bacterial membrane proteins such as OMPCs from Neisseria meningitidis (Vella and al., Infect. Immun., 60, 1992, 4977-4983), TraT from Escherichia coli (Croft and al., J. Immunol., 146, 1991, 793-798) or PorB from Neisseria meningitidis (Fusco and al., J. Infect. Dis., 175, 1997, 364-372).

Furthermore, antigens generally possess low immunogenicity and it is necessary to mix them with adjuvants.

Thus, a great need now exists to have a compound which, combined with a molecule, in particular an antigen or a hapten, is capable of generating an immune response directed against said molecule. Such a compound could in particular be used for the preparation of a vaccine composition intended to induce antiviral, antibacterial, antifungal or antiparasitic, or antitumor immune protection.

OmpA from Klebsiella pneumoniae, a major outer membrane protein called P40, has a carrier protein activity, by the systemic route, for peptide subunit antigens (patents WO 95/27787 and WO 96/14415; Haeuw and al., Eur. J. Biochem., 255, 1998, 446-454; Plotnicky-Gilquin and al., J. Virol., 73, 1999, 5637-5645) and polysaccharide subunit antigens (patent WO 97/41888; Rauly and al., Infect. Immun., 67, 1999, 5547-5551).

This activity, which is due to the presence of at least one T epitope on the protein (Haeuw and al.) is comparable to that of a reference carrier protein such as the tetanus toxoid (Rauly and al.), a protein used in vaccines for human use.

A structural model of d'Escherichia coli OmpA has been established on the basis of biochemical and physicochemical studies (Ried and al., Mol. Gen. Genet., 243, 1994, 127-135; Koebnik and Krämer, J. Mol. Biol., 250, 1995, 617-626; Pautsch and Schulz, Nature Struct. Biol., 5, 1998, 1013-1017). According to this model, the OmpAs are thought to have two distinct domains: a membrane domain having a β-pleated sheet structure, and a periplasmic domain without a defined structure.

There may be mentioned more particularly the document patent WO 96/14415 which describes the complete P40 protein of K. pneumoniae, its three transmembrane fragments AA 1-179, AA 108-179 and AA 127-179 and their use for the preparation of a vaccine, adjuvant composition or of an immunogenic complex. There may also be mentioned the Puohiniemi et al. document (Infection and Immunity, 58(6), 1691-1696, 1990) which describes, in particular, the antibody response produced in mice after immunization with a complete OmpA protein from enterobacteria or with whole enterobacteria (such as E. coli and Salmonella typhimurium) and the Nguyen et al. document (Gene, 210, 93-101, 1998) which describes a particular PCR method used for the isolation and complete sequencing of the gene encoding K. pneumoniae OmpA.

The main disadvantage linked to the use of membrane proteins is due to the need to use detergents in order to solubilize them and to maintain them in solution, these detergents then having to form part of the final vaccine formulation.

Thus, when such proteins are intended in particular to be injected in vivo, the presence of these detergents is potentially toxic (risk of necrosis, and the like).

A need therefore now exists to have a carrier protein and/or an adjuvant capable of generating an immune response in the absence or in an insignificant quantity of detergent.

This is precisely the subject of the present invention.

Surprisingly, it has been demonstrated that a peptide obtained from a periplasmic domain or an enterobacterium Omp protein, in particular an enterobacterium OmpA protein such as the Klebsiella pneumoniae P40 protein, satisfies these needs.

The subject of the present invention is thus the use of the periplasmic domain of an enterobacterium Omp protein free of the membrane part, as carrier protein and/or adjuvant by the systemic route in order to increase the immunogenicity of an antigen which is covalently attached, or which is chemically conjugated (in particular by means of a coupling reagent), or which is fused (chimeric protein expressed by genetic recombination when the combined antigen is of a protein nature). This periplasmic fragment lacking the membrane domain of the protein is active in the absence of a detergent, unlike the full-length protein. Its activity is furthermore equivalent to that of the full-length protein used in the presence of a detergent.

In the present invention, the term “peptide” will also be understood to mean polypeptides and proteins, these three terms being used interchangeably, and the term “Omp” will be understood to mean outer membrane protein.

The expression peptide obtained from a periplasmic domain of an enterobacterium Omp protein is understood to denote, in particular, any peptide whose amino acid sequence is included in the amino acid sequence of a periplasmic domain of an enterobacterium Omp protein and which, when administered to an organism, in particular to an animal or to humans, is capable of generating or increasing an immune response, in particular directed against an infectious agent or a tumor cell when said periplasmic peptide is combined with an antigen or a hapten specific for said infectious agent or for said tumor cell.

The subject of the invention is a pharmaceutical composition, characterized in that it comprises, in a pharmaceutically acceptable medium, at least one peptide obtained from a periplasmic domain of an enterobacterium Omp protein or a nucleic construct encoding said peptide.

The expression nucleic construct encoding said peptide obtained from a periplasmic domain of an enterobacterium Omp protein is understood to mean preferably a nucleic construct, in particular of the DNA or RNA type, in which the nucleic acid encoding a peptide derived from an enterobacterium Omp protein consists exclusively of a nucleic acid encoding its periplasmic domain, one of its homologous peptides or one of its fragments as defined below for the terms homology or fragments.

Of course, said nucleic construct may, in addition, comprise other nucleic sequences necessary, for example, for the expression or the cloning of said peptide obtained from a periplasmic domain of an enterobacterium Omp protein (nucleic construct of an expression and/or cloning vector), and/or a nucleic sequence encoding the immunogen, antigen or hapten which it is desired to combine with said peptide derived from the Omp protein.

The expression peptide obtained from a periplasmic domain of an enterobacterium Omp protein is understood in particular to mean a peptide chosen from:

a periplasmic domain of an enterobacterium Omp protein;

a peptide exhibiting a homology, preferably an identity after optimum alignment, of at least 80%, preferably 85%, 90%, 95% and 99% with a periplasmic domain of an enterobacterium Omp protein;

a peptide of at least 5 consecutive amino acids, preferably at least 8, 10, 15, 20, 25, 30, 40, 45 and 50 consecutive amino acids of a periplasmic domain of an enterobacterium Omp protein and exhibiting immunogenic activity; and

a helper T epitope of a periplasmic domain of an enterobacterium Omp protein.

The periplasmic domain of an enterobacterium Omp protein may be obtained by a method of extraction from a culture of said enterobacterium.

Methods for extracting bacterial membrane proteins are known to persons skilled in the art and will not be developed in the present description. There may be mentioned, for example, but without limitation, the method of extraction described by Haeuw J. F. et al.

The periplasmic domain of an enterobacterium Omp protein may also be obtained by the recombinant route.

Methods for preparing recombinant proteins are now well known to persons skilled in the art and will not be developed in the present description; reference will be made however to the method described in the examples. Among the cells which can be used for producing these recombinant proteins, there should of course be mentioned bacterial cells (Olins P. O. and Lee S. C., 1993, Curr. Op. Biotechnology, 4:520-525), but also yeast cells (Buckholz R. G., Curr. Op. Biotechnology, 4:538-542, 1993), as well as animal cells, in particular mammalian cell cultures (Edwards C. P. and Aruffo A., Curr. Op. Biotechnology, 4:558-563, 1993) but also insect cells in which it is possible to use methods using, for example, baculoviruses (Luckow V. A., Curr. Op. Biotechnology, 4:564-572, 1993).

Most preferably, the peptide obtained from a periplasmic domain of an enterobacterium Omp protein is chosen from a peptide obtained from a periplasmic domain of an OmpA protein, and more preferably from a peptide obtained from the periplasmic domain of a Klebsellia pneumoniae OmpA protein.

In another preferred embodiment of the invention, the peptide obtained from a periplasmic domain of an enterobacterium Omp protein comprises, preferably consists of, a peptide whose sequence is chosen from the following sequences:

a) the amino acid sequence having the sequence SEQ ID No. 2;

b) the amino acid sequence of a sequence exhibiting a homology, preferably an identity after optimum alignment, of at least 80%, preferably 85%, 90%, 95% and 99% with the sequence SEQ ID No. 2; and

c) the amino acid sequence of a fragment of at least 5 amino acids, preferably of at least 8, 10, 15, 20, 25, 30, 40, 45 and 50 consecutive amino acids of a sequence as defined in a).

The expression nucleic acid or amino acid sequence exhibiting a homology of at least 80% after optimum alignment with a determined nucleic acid or amino acid sequence is understood to denote a sequence which, after optimum alignment with said determined sequence, exhibits a percentage identity of at least 80% with said determined sequence.

The expression “percentage identity” between two nucleic acid or amino acid sequences for the purposes of the present invention is understood to denote a percentage of nucleotides or of amino acid residues which are identical between the two sequences to be compared, which is obtained after the best alignment, this percentage being purely statistical and the differences between the two sequences being randomly distributed over the entire length. Sequence comparisons between two nucleic acid or amino acids sequences are traditionally carried out by comparing these sequences after having optimally aligned them, said comparison being performed per segment or per “comparison window” in order to identify and compare the local regions with sequence similarity. The optimum alignment of the sequences for comparison may be performed, other than manually, by means of the Smith and Waterman local homology algorithm (Ad. App. Math., 2:482-489, 1981), by means of the Neddleman and Wunsch local homology algorithm (J. Mol. Biol., 48:443-453, 1970), by means of the Pearson and Lipman search for similarity method (Proc. Natl. Acad. Sci., USA, 85:2444, 1988), by means of computer software packages using these algorithms (GAP, BESTFIT, FASTA and TFASTA in the Wisconsin Genetics Software Package, Genetics Computer Group, 575 Science Dr., Madison, Wis., or by the BLAST N or BLAST P comparison software packages).

The percentage identity between two nucleic acid or amino acid sequences is determined by comparing these two sequences optimally aligned by a comparison window in which the region of the nucleic acid or amino acid sequence to be compared may comprise additions or deletions relative to the reference sequence for an optimum alignment between these two sequences. The percentage identity is calculated by determining the number of identical positions for which the nucleotide or the amino acid residue is identical between the two sequences, by dividing this number of identical positions by the total number of positions in the comparison window and multiplying the result obtained by 100 in order to obtain the percentage identity between these two sequences.

For example, it will be possible to use the BLAST program, “BLAST 2 sequences” available at the site http://www.ncbi.nlm.nih.gov/gorf/bl2.html, the parameters used being those defined by default (in particular for the “open gap penalty”: 5, and “extension gap penalty” parameters: 2; the matrix chosen being for example the “BLOSUM 62” matrix proposed by the program), the percentage identity between the two sequences to be compared being directly calculated by the program.

Among said sequences allowing a homology of at least 80% with the reference OmpA sequence or among the sequences of a fragment of the periplasmic domain of the reference OmpA, those preferred are the sequences of, or encoding, peptides capable of inducing an immune response specifically directed against the antigen or hapten with which it is combined, such as the induction of an immune response measured by means of the standard techniques described in the examples below.

The present invention also relates to a composition according to the invention, characterized in that the pharmaceutical composition comprises a nucleic construct encoding said peptide obtained from a periplasmic domain of an enterobacterium Omp protein or a transformed host cell capable of expressing said peptide obtained from a periplasmic domain of an enterobacterium Omp protein.

The subject of the invention is also the composition according to the invention, characterized in that said pharmaceutical composition comprises, in addition, an antigen, an immunogen or a hapten.

The expression “immunogen, antigen or hapten specific for an infectious agent or a tumor cell” is understood to denote in particular any compound expressed by an infectious agent, such as a virus, a bacterium, a yeast, a fungus or a parasite, by a tumor cell, or one of their structural analogs, which, alone or in combination with an adjuvant or a carrier, is capable of inducing an immune response specific for said infectious agent or said tumor cell.

The expression “immunogen, antigen or hapten” is also understood to mean in the present description a compound exhibiting a structural analogy with said antigen or hapten capable of inducing an immunological response directed against said antigen or hapten in an organism immunized beforehand with said analogous compound.

Said antigen or hapten may in particular be chosen from proteins, glycopeptides, lipopeptides, polysaccharides, oligosaccharides, nucleic acids and lipids.

In one embodiment of the invention, said antigen, immunogen or hapten is derived from a virus, a bacterium, a parasite or a fungus.

In a preferred embodiment of the invention, said antigen, immunogen or hapten comprises at least one peptide derived from a microorganism responsible for pathologies of the airways, chosen from RSV (respiratory syncytial virus), parainfluenza virus (PIV), influenza virus, hantaviruses, streptococci, pneumococci, haemophilus influenza type b, rhinoviruses, coronoviruses and meningococci.

In a still more preferred embodiment of the invention, said antigen, immunogen or hapten comprises at least one fragment of the G protein of the respiratory syncytial virus type A or B, in particular of human origin.

In another embodiment of the invention, said antigen, immunogen or hapten is combined or specific for a tumor cell.

Among the cancers whose tumors express an associated tumor antigen which can be prevented or treated by the uses according to the present invention, there may be mentioned in particular, but without limitation:

breast cancer, lung cancer, colon cancer, and gastric carcinoma (Kawashima et al., Cancer Res., 59:431-435, 1999);

mesothelioma, osteosarcoma, brain cancers (Xie et al., J. Natl. Cancer. Inst., 91:169-175, 1999);

melanoma (Zheuten et al., Bratilsl. Lek. Listy, 99:426-34, 1998);

cystic adenoma of the pancreas (Hammel et al., Eur. J. gastroenterol. Hepatol., 10:345-348, 1998);

colorectal cancer (Ogura et al., Anticancer Res., 18:3669-3675, 1998);

kidney cell carcinoma (Jantzer et al., Cancer Res., 58:3078-3086, 1998); and

ovarian and cervical cancer (Sonoda et al., Cancer., 77:1501-1509, 1996).

The subject of the present invention is also a composition according to the invention, characterized in that said antigen, immunogen or hapten is coupled or mixed with the peptide obtained from a periplasmic domain of an enterobacterium Omp protein.

The invention also comprises the use according to the invention, characterized in that said antigen or hapten is coupled by covalent bonding, in particular by chemical bonding, to said peptide obtained from a periplasmic domain of an enterobacterium Omp protein.

In a particular embodiment of the invention, there are introduced one or more linking components, said peptide being obtained from a periplasmic domain of an Omp protein, or one of its fragments, and/or in said antigen or hapten in order to facilitate chemical coupling, preferably said linking component introduced is an amino acid.

According to the invention, it is possible to introduce one or more linking elements, in particular amino acids in order to facilitate coupling reactions between the peptide obtained from a periplasmic domain of an enterobacterium Omp protein, and said immunogen, antigen or hapten. The covalent coupling between the peptide obtained from a periplasmic domain of an enterobacterium Omp protein and said immunogen, antigen or hapten according to the invention may be performed at the N- or C-terminal end of said peptide. The bifunctional reagents allowing this coupling will be determined according to the end of said peptide which is chosen in order to carry out the coupling and the nature of said immunogen, antigen or hapten to be coupled.

In another particular embodiment, the use according to the invention is characterized in that the coupling between said immunogen, antigen or hapten and said peptide obtained from a periplasmic domain of an enterobacterium Omp protein is carried out by genetic recombination, when said immunogen, antigen or hapten is of a peptide nature.

The conjugates derived from a coupling between the immunogen, antigen or hapten and said peptide obtained from a periplasmic domain of an enterobacterium Omp protein, may be prepared by genetic recombination. The chimeric or hybrid protein (the conjugate) may be produced by recombinant DNA techniques by insertion or addition to the DNA sequence encoding said peptide obtained from a periplasmic domain of an enterobacterium Omp protein, of a sequence encoding said immunogen, antigen or hapten of a protein nature.

The method for synthesizing the hybrid molecules include the methods used in genetic engineering to construct hybrid polynucleotides encoding the desired polypeptide sequences. Reference may advantageously be made, for example, to the technique for producing genes encoding fusion proteins which is described by D. V. Goeddel (Gene expression technology, Methods in Enzymology, vol. 185, 3-187, 1990).

Accordingly, the present invention also relates to a pharmaceutical composition which comprises a nucleic construct encoding said hybrid protein, or which comprises a vector containing a nucleic construct encoding said hybrid protein or a transformed host cell containing said nucleic construct capable of expressing said hybrid protein.

The compositions according to the invention may in addition contain an adjuvant. The latter may in particular be chosen from MPL-A (“MonoPhosphoryl Lipid A”), Quil-A (adjuvant derived from saponin), ISCOM (“ImmunoStimulating COMplex”), Dimethyl Dioctadecyl Ammonium in bromide form (DDAB) or in chloride form (DDAC), CpGs (oligodeoxynucleotides containing a specific unit centered around a dinucleotide CpG), Leif (protein antigen derived from Leishmania capable of stimulating PBMC cells and antigen-presenting cells, and of producing a cytokine reaction of the Th-1 type), CT (Cholera Toxin), LT (“heat Labile Toxin”) and detoxified versions of CT or LT.

In a preferred embodiment of the invention, the pharmaceutical composition according to the invention contains no adjuvant other than the peptide obtained from a periplasmic domain of an enterobacterium Omp protein.

In a still more preferred embodiment of the invention, said pharmaceutical composition according to the invention contains no detergent.

For the purposes of the present invention, the pharmaceutically acceptable medium is the medium in which the compounds of the invention are administered, preferably a medium which is injectable to humans. It may consist of water, an aqueous saline solution or an aqueous solution based on dextrose and/or glycerol.

The invention also comprises a composition according to the invention, characterized in that said pharmaceutical composition is transported in a form which makes it possible to improve its stability and/or its immunogenicity; thus, it may be transported in the form of liposomes, virosomes, nanospheres, microspheres or microcapsules.

The present invention further relates to a protein of the periplasmic domain of an enterobacterium Omp chosen from proteins having the following sequences:

a) the protein having the sequence SEQ ID No. 2;

b) a protein having a sequence exhibiting a homology, preferably an identity after optimum alignment, of at least 80%, preferably 85%, 90%, 95% and 99% with the sequence SEQ ID No. 2;

c) a protein of at least 5 amino acids, preferably of at least 8, 10, 15, 20, 25, 30, 40, 45, 50, 75 and 100 consecutive amino acids of the periplasmic domain having the sequence SEQ ID No. 2 and exhibiting immunogenic activity, in particular as a carrier protein and/or as an adjuvant.

The present invention also relates to a nucleic construct, in particular of the DNA type, encoding the periplasmic domain of an enterobacterium Omp protein having the sequence SEQ ID No. 2, its homologous sequences or its fragments as defined above in b) and c).

The present invention relates in particular to a nucleic construct, characterized in that the nucleic acid encoding the peptide obtained from the periplasmic domain is chosen from the amino acids having the following sequence:

a) the nucleic acid having the sequence SEQ ID No 1;

b) a nucleic acid having the sequence exhibiting a homology, preferably an identity after optimum alignment, of at least 80%, preferably 85%, 90%, 95% and 99% with the sequence SEQ ID No. 1; and

c) a nucleic acid whose sequence consists of the sequence of a fragment of at least 15 nucleotides, preferably 24, 30, 45, 60, 75, 90, 105, 120, 135, 150, 225 and 300 consecutive nucleotides of the sequence SEQ ID No. 1 and whose peptide encoded by said fragment exhibits an immunogenic activity, in particular as a carrier protein and/or as an adjuvant.

The sequence SEQ ID No. 2 corresponds to amino acids 199 to 335 of Klebsiella pneumoniae OmpA (OmpA in which the protein of 335 amino acids is also called protein P40). This fragment lacks the first 17 amino acids of the periplasmic domain (amino acids 182 to 198 of said sequence of Klebsiella pneumoniae OmpA, in order to remove the so-called “hinge” zone, which exhibits a strong homology with the light chain 1 of human myosin.

The use of a peptide obtained from a periplasmic domain of an enterobacterium Omp protein as defined above as a carrier and/or an adjuvant in a vaccine (or for the preparation of a vaccine) constitutes another subject of the invention.

The invention also relates to the use of a peptide obtained from a periplasmic domain of an enterobacterium Omp protein as defined above for the preparation of a vaccine intended for the prophylactic or therapeutic treatment of viral, bacterial, parasitic or fungal infections or for the preparation of a vaccine intended for the prophylactic or therapeutic treatment of cancers.

The subject of the invention is also the use of a peptide obtained from a periplasmic domain of an enterobacterium Omp protein as defined above for the preparation of a pharmaceutical composition intended to generate or increase an immune response against an infectious agent or a tumor cell.

The legend to the figures and examples which follow is intended to illustrate the invention without at all limiting the scope thereof.

Legend to the Figures:

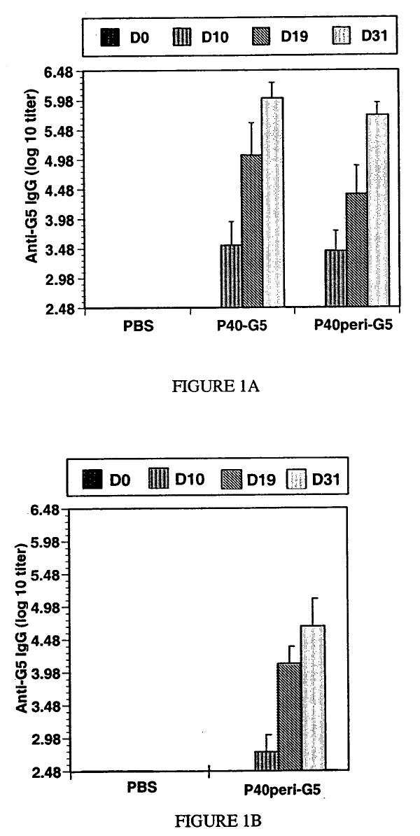

FIGS. 1A and 1B: Study of the serum anti-G5 response after immunization of BALB/c mice with the conjugates P40-G5 and P40peri-G5 (“P40peri” for the fragment of the periplasmic domain of the P40 protein).

### EXAMPLE 1

### Cloning of the Periplasmic Domain of P40

The gene encoding the periplasmic portion was obtained by PCR amplification from plasmid DNA encoding rP40 using, as pair of primers:

| | |
|---|---|
| the sense oligonucleotide having the sequence: | |
| 5′-GCGAATTCGTGGCTACCAAGCACTTCACC-3′; and | (SEQ ID No. 3) |
| the antisense oligonucleotide having the sequence: | |
| 5′-CGAAGCTTAGTGGTGGTGGTGGTGGTGTTCCGGAGCCGGAGCCG-3′. | (SEQ ID No. 4) |

The fragment thus amplified, corresponding to P40peri (“P40peri” or “P40p” for the fragment of the periplasmic domain of the P40 protein) and identified by sequencing, was cloned into a vector under the control of the tryptophan operon promoter, upstream of 9 amino acids of the leader peptide (MKAIFVLNA (SEQ ID No. 5)). The protein sequence of the periplasmic part corresponds to amino acids 182 to 335 of Klebsiella pneumoniae OmpA.

### EXAMPLE 2

### Expression of the Periplasmic Domain of P40 in Escherichia coli

The Escherichia coli ICONE 100 strain (ICONE 100 strain as described in application WO 99/53080 published on 15 Oct. 1999) transformed with the vector pvaLP40p is cultured overnight at 37° C., with shaking, in 100 ml of TSB supplemented with yeast extract, with tetracycline (8 μg/ml). The next day, a culture, diluted to a biomass concentration equivalent to an optical density OD=1 for a wavelength of 580 nm, is prepared in TSB medium supplemented with yeast extract, tetracycline and tryptophan (100 μg/ml). After 10 minutes of culture, the expression of the recombinant protein is induced by addition of IAA (indole-3-acrylic acid) at 25 μg/ml in the medium. About 5 hours at 37° C. after induction, the cells are harvested by centrifugation.

### EXAMPLE 3

### Extraction and Purification of the Periplasmic Domain of P40

After centrifugation, the cells are resuspended in a 25 mM Tris-HCl buffer, pH 8.5, and then treated with lysozyme (0.5 g/litre for 1 hour at room temperature, with gentle stirring). After centrifugation (25 min at 10 000 g and at +4° C.), the supernatant is dialyzed overnight at +4° C., with stirring, against a 20 mM sodium phosphate buffer, pH 7, containing 0.5 M NaCl.

The purification is carried out by affinity chromatography on immobilized metal. The preceding dialysate is deposited on a Chelating Sepharose type column (Amersham Pharmacia Biotech) previously charged with Ni²⁺ ions with the aid of a 1 M NiSO₄ solution. The protein is eluted with the aid of an imidazole gradient from 0 to 0.5 M.

The protein may be concentrated, after purification, by tangential ultrafiltration.

1 liter of culture makes it possible to obtain 50 to 100 mg of purified protein.

The protein concentration is determined by assaying using the BCA method (protein microassay with BicinChoninic Acid), and the purity of the purified protein is determined by analysis by SDS-Page electrophoresis. The purified periplasmic domain has an apparent molecular mass of 18 kDa.

### EXAMPLE 4

### Coupling of the G5 Peptide

The G5 peptide (SEQ ID. No. 6) is a synthetic peptide of 16 amino acids, whose sequence is the following:

Ser Lys Pro Thr Thr Lys Gln Arg Gln Asn Lys Pro Pro Asn Lys Pro
1 5 10 15

This peptide corresponds to the part 144-159 of the G protein of the respiratory syncytial virus.

It is coupled to the proteins P40 and P40peri, by means of a cysteine residue introduced at the C-terminal position, with the aid of the bromo-N-hydroxysuccinimide acetate reagent according to the method described by Haeuw et al.

The conjugates obtained are analyzed by SDS-PAGE (PolyAcrylamide Gel Electrophoresis) electrophoresis. The level of coupling of the peptide to the proteins is estimated by assaying the carboxymethylcysteine residue released after hydrolysis (6N HCl) of the conjugates.

The level of coupling determined by this method is about 3 to 5 mol of peptides coupled per mole of P40peri.

### EXAMPLE 5

### Immunogenicity of the P40peri-G5 Conjugate

The periplasmic part of the P40 protein was compared with the full-length P40 for its capacity to induce an antibody response against a pure B epitope: the G5 peptide described above.

The extraction and the purification of the rP40 (rP40 for recombinant protein P40) were described by Haeuw et al., and in Applications WO 95/27787 and FR 00 02 104.

For this study, the G5 peptide was coupled according to the method indicated above to the P40 and P40peri proteins. 10 μg of G5 equivalents were administered, by the subcutaneous route, A in the presence or B in the absence of aluminum hydroxide to BALB/c mice (n=5), at D0, D10 and D20. Individual needle biopsy was performed on the mice before each immunization and 11 days after the last immunization, and the serum anti-G5 IgG titer was determined by ELISA.

The results presented in FIGS. 1A and 1B show that the periplasmic portion of P40 is capable, like the full-length P40, of inducing an antibody response directed against a pure B epitope in the presence or in the absence of adjuvant.

In the presence of adjuvant (FIG. 1A), the intensity of the anti-G5 response and the kinetics of response are identical in both cases.

In the absence of adjuvant (FIG. 1B), an anti-G5 response is noted when G5 is conjugated with P40peri.

These results demonstrate the carrier role of the periplasmic portion of the P40 protein.

1/ A pharmaceutical composition, characterized in that it comprises, in a pharmaceutically acceptable medium, at least one peptide of at least 10 amino acids obtained from a periplasmic domain of an enterobacterium Omp protein or a nucleic construct encoding said peptide, said peptide having as sequence an amino acid sequence comprised in the amino acid sequence of said periplasmic domain.

2/ The composition as claimed in claim 1, characterized in that the peptide obtained from a periplasmic domain of an enterobacterium Omp protein is chosen from the following peptides:
a peptide of at least 10 consecutive amino acids of a periplasmic domain of an enterobacterium Omp protein and exhibiting immunogenic activity; and
a helper T epitope of a periplasmic domain of an enterobacterium Omp protein.

3/ The composition as claimed in claim 1 or 2, characterized in that the peptide obtained from a periplasmic domain of an enterobacterium Omp protein is chosen from a peptide obtained from a periplasmic domain of an Omp A protein and more preferably from a peptide obtained from a periplasmic domain of a Klebsellia pneumoniae Omp A protein.

4/ The composition as claimed in any one of claims 1 to 3, characterized in that the peptide obtained from a periplasmic domain of an enterobacterium Omp protein consists of a peptide whose sequence is chosen from the following sequences:
a) the amino acid sequence having the sequence SEQ ID No. 2;
b) the amino acid sequence of a sequence exhibiting a homology of at least 80% with the sequence SEQ ID No. 2; and
c) the amino acid sequence of a fragment of at least 10 consecutive amino acids of a sequence as defined in a).

5/ The composition as claimed in any one of claims 1 to 4, characterized in that it contains, in addition, an antigen, an immunogen or a hapten.

6/ The composition as claimed in claim 5, characterized in that said antigen, immunogen or hapten is chosen from peptides, glycopeptides, lipopeptides, poly- or oligosaccharides, nucleic acids and lipids.

7/ The composition according to claim 5 or 6, characterized in that said antigen, immunogen or hapten is derived from a virus, a bacterium, a parasite or a fungus.

8/ The composition as claimed in claim 7, characterized in that said antigen, immunogen or hapten comprises at least one peptide derived from a microorganism responsible for pathologies of the airways which is chosen from the RSV, the parainfluenza virus (PIV), the influenza virus, hantaviruses, streptococci, pneumococci, haemophilus influenza type b, rhinoviruses, coronoviruses and meningococci.

9/ The composition as claimed in claim 7 or 8, characterized in that said antigen, immunogen or hapten comprises at least one fragment of the G protein of the respiratory syncytial virus.

10/ The composition as claimed in claim 5, characterized in that the antigen, immunogen or hapten is combined or specific for a tumor cell.

11/ The composition as claimed in one of claims 5 to 10, characterized in that said antigen, immunogen or hapten is combined, by mixing or by coupling, with the peptide obtained from a periplasmic domain of an enterobacterium Omp protein.

12/ The composition as claimed in claim 11, characterized in that the coupling between said antigen, immunogen or hapten and said peptide obtained from a periplasmic domain of an enterobacterium Omp protein is carried out by genetic recombination.

13/ The composition as claimed in one of claims 1 to 12, characterized in that said pharmaceutical composition contains, in addition, an adjuvant, preferably chosen from the group of adjuvant comprising MPL-A, Quil-A, ISCOM, Dimethyl Dioctadecyl Ammonium in bromide form (DDAB) or in chloride form (DDAC), CpGs, Leif, CT, LT and the detoxified versions of CT or LT.

14/ The composition as claimed in one of claims 1 to 12, characterized in that said pharmaceutical composition contains no adjuvant other than the peptide obtained from a periplasmic domain of an enterobacterium Omp protein.

15/ The composition as claimed in one of claims 1 to 14, characterized in that said composition contains no detergent.

16/ The composition as claimed in one of claims 1 to 15, characterized in that said pharmaceutically acceptable medium consists of water, an aqueous saline solution or an aqueous solution based on dextrose and/or glycerol.

17/ The composition as claimed in one of claims 1 to 16, characterized in that said pharmaceutical composition is transported in a form which makes it possible to improve its stability and/or its immunogenicity.

18/ A peptide obtained from the periplasmic domain of an enterobacterium Omp chosen from the peptides having the following sequence:
a) the peptide having the sequence SEQ ID No. 2;
b) a peptide having the sequence exhibiting a homology of at least 80% with the sequence SEQ ID No. 2;
c) a peptide of at least 10 consecutive amino acids of the periplasmic domain having the sequence SEQ ID No. 2 and exhibiting immunogenic activity, in particular as a carrier protein and/or as an adjuvant.

19/ A nucleic construct encoding a peptide as claimed in claim 18.

20/ The nucleic construct as claimed in claim 19, characterized in that the nucleic acid encoding the peptide obtained from the periplasmic domain is chosen from the nucleic acids having the following sequence:
a) the nucleic acid having the sequence SEQ ID No. 1;
b) a nucleic acid whose sequence consists of the sequence of a fragment of at least 30 consecutive nucleotides having the sequence SEQ ID No. 1 and whose peptide encoded by said fragment has immunogenic activity, in particular as a carrier protein and/or as an adjuvant.

21/ The use of a peptide obtained from a periplasmic domain of an enterobacterium Omp protein as defined in one of claims 1 to 4 as a carrier and/or an adjuvant for the preparation of a vaccine.

22/ The use of a peptide obtained from a periplasmic domain of an enterobacterium Omp protein as defined in one of claims 1 to 4 for the preparation of a vaccine intended for the prophylactic or therapeutic treatment of viral, bacterial, parasitic or fungal infections or for the preparation of a vaccine intended for the prophylactic or therapeutic treatment of cancers.

23/ The use of a peptide obtained from a periplasmic domain of an enterobacterium Omp protein as defined in one of claims 1 to 4 for the preparation of a pharmaceutical composition intended to generate or increase an immune response against an infectious agent or a tumor cell.
